(19) United States
(12) Patent Application Publication
(10) Pub. No.: US 2025/0073246 A1
(43) Pub. Date: Mar. 6, 2025

(54) METHOD OF TREATING HYPOGONADISM IN MEN

(71) Applicant: Shane Kelleher, Fort Worth, TX (US)

(72) Inventor: Shane Kelleher, Fort Worth, TX (US)

(21) Appl. No.: 18/239,522

(22) Filed: Aug. 29, 2023

(51) Int. Cl. A61K 31/569; A61K 31/4196; A61K 31/616; A61P 15/08; G01N 33/74

(52) U.S. Cl. CPC A61K 31/569; A61K 31/4196; A61K 31/616; A61P 15/08; G01N 33/743; G01N 2333/805; G01N 2800/344; G01N 2800/52

(57) ABSTRACT

A method of treating hypogonadism in a human male patient comprises the steps of administering a therapeutically effective amount of a testosterone androstane steroid derivative (TASD) such as testosterone cypionate sufficient to maintain a total serum testosterone level within a therapeutic/stabilized range. A therapeutically effective amount of an aromatase inhibitor, such as anastrozole, sufficient to maintain an estradiol level at or below 35 pg/mL and an estrogen level above a predetermined level may be administered. A therapeutically effective amount of aspirin is administered if the patient is determined to have an elevated hemoglobin level. In certain embodiments, the hemoglobin level is determined to be elevated if the hemoglobin level is greater than about 16 g/dL and blood is removed from the patient, in therapeutic amounts, if the hemoglobin level is greater than 17 g/dL. In certain embodiments, the serum total testosterone level therapeutic/stabilized range is between about 605-1051 ng/dL.

### BACKGROUND OF THE INVENTION

### 1. Field of the Invention

The present invention relates generally to a hormone treatment protocol and specifically to a method of treating hypogonadism in men.

### 2. Description of the Prior Art

Testosterone is a male sex hormone produced primarily in the testes. While often associated with sexual health, testosterone plays a critical role in a range of physiological processes. For example, testosterone influences bone density, red blood cell production, mood regulation, and overall energy levels. Thus, testosterone extends beyond sexual health, impacting various aspects of a man's well-being.

Hypogonadism (low total testosterone (TT) levels) is an increasingly prevalent concern affecting men of various age groups. For example, over the last 15-20 years, TT levels have been dropping across the United States. Factors contributing to this decline include irregular work schedules, processed food intake, stress, and many others. Many professions, for example, police officers, firefighters, medical professionals, and business owners suffer from low TT levels. This is likely due to the high stress and irregular schedules associated with their careers.

Low TT levels can manifest in a variety of ways, including reduced libido, erectile dysfunction, decreased muscle mass, fatigue, mood changes, and cognitive difficulties.

Diagnosing low TT levels involves a comprehensive evaluation of symptoms, medical history, physical examination, and laboratory tests measuring serum testosterone levels. The decline in testosterone levels has led to a significant increase in patients treated with testosterone replacement therapy (TRT). A number of formulations, treatments, and therapies related to TRT are known in the art. For example, Amory, U.S. Pat. No. 7,138,389 discloses an oral androgen therapy formulated in an oil vehicle.

Betageri, U.S. Pat. Nos. 8,957,053 and 9,445,995 provide a proliposomal testosterone powder formulation for oral dosage in hypogonadal patients.

Wotton, U.S. Pat. No. 9,950,125 provides a method for subcutaneous injection testosterone treatment.

Glaser, U.S. Pat. No. 10,792,290 teaches a subcutaneous pellet implant pharmaceutical composition containing testosterone and an aromatase inhibitor.

Lagomasino, US Pat. Pub. No. 20140171918, discloses a subcutaneous injection technique to deliver testosterone in hypogonadal men.

Dudley, US Pat. Pub. No. 20150250801 provides an androgen pharmaceutical composition and method for treating depression delivered percutaneously through the synthetic pathway.

Dhingra, US Pat. Pub. No. 20220265678 provides an oral testosterone undecanoate therapy method.

However, conventional testosterone replacement therapy often results in undesirable side effects often seen when testosterone levels fluctuate in the patient's body. These side effects include, but are not limited to, acne, elevated estradiol levels (due to conversion of testosterone cypionate (TC) to estradiol), hair loss, aggression, and polycythemia. Polycythemia is a blood disorder related to excess hemoglobin levels. Polycythemia causes an increased risk for blood clotting, stroke or heart attack. Polycythemia can also cause numerous other symptoms such as fatigue, weakness, headache, dizziness, shortness of breath, and high blood pressure. Polycythemia associated with TRT has commonly been controlled through frequent patient blood donation.

What is needed is a method of treating hypogonadism in men that permits a clinician to identify an appropriate level of hormone replacement dosing that maximizes benefits to the patient while minimizing side effects such as polycythemia.

### BRIEF SUMMARY OF THE INVENTION

### General Summary

The method for treating hypogonadism in men of the preferred embodiment generally comprises the steps of: determining whether a patient has presenting symptoms consistent with low testosterone; determining a total testosterone level (“TT”) of the patient; if the TT level is below about 500 ng/dL, administering to the patient about 140 mg Testosterone Cypionate (“TC”) injections about q7 (about every 7 days); about one week after the first four consecutive testosterone replacement injections (after four consecutive TC injections), determining the patient's TT, CBC (complete blood count), and estradiol (E2) levels; determining whether the patient has symptoms consistent with elevated estrogen levels, (if the patient has bloating, water weight gain, or E2 levels above about 35 μg/mL), starting the patient on about 1 mg anastrozole, once weekly by mouth, and monitoring the patient for complaints of low estrogen levels (hot flashes and/or new joint pains), and if there are no such symptoms, continuing the patient with about the same dose of anastrozole, and if there are such symptoms consistent with low estrogen levels, decreasing the anastrozole dosage to about 0.5 mg, once weekly; if the TT is between about 605-1051 ng/dL (“therapeutic/stabilized range”) and the presenting symptoms have improved, continuing the patient on about the 140 mg TC therapy; if the TT is between about 605-1051 ng/dL but the presenting symptoms have not improved, assessing the E2, TT, and hemoglobin levels and considering other sources and/or causes of the symptoms; if the TT is above about 1051 ng/dL, adjusting the TC dosage downward about 20 mg; if less than about 605 ng/dL, adjusting the TC dosage upward about 20 mg; continuing to monitor the patient periodically, for example, about once every six to twelve months, and repeating the steps listed above beginning with “about one month after the first injection . . . ”.

The method for treating hypogonadism in men of preferred embodiments is used to achieve “Stabilization”. In the method of the preferred embodiment, the first step begins with determining whether the patient has presenting symptoms consistent with low testosterone. In preferred embodiments, the symptoms that are consistent with low testosterone include fatigue, poor motivation, decreased libido, poor sleep quality, weight gain, mental fogginess (lack of mental clarity), moodiness, depression, loss of muscle mass, and erectile dysfunction.

If the patient has symptoms consistent with low testosterone, the next step is to determine the patient's total testosterone level. Although in the preferred embodiment, the patient's total testosterone is measured through a total testosterone test, in other embodiments, other tests may be used to determine the patient's level of circulating testosterone and Stability. It is preferable that the respective methods employed to measure the initial total testosterone and other levels be consistent with the respective methods used to monitor and re-measure total testosterone and other levels prior to and during testosterone therapy.

In other embodiments of the method, additional blood work is performed at the time the initial total testosterone levels are measured. In such embodiments, the testing comprises one or more tests to determine the patient's estradiol (E2) level, thyroid stimulating hormone (TSH) level, prostate-specific antigen (PSA) level, CBC, A1c, and/or vitamin D level. These additional studies may be used by the clinician, for example, to determine whether any of the patient's presenting symptoms result from other conditions.

After the step of measuring the patient's total testosterone level, the next step of the method of the preferred embodiment is to start the patient on about 140 mg testosterone cypionate (“TC”) injections about q7 (about every 7 days) if the patient's TT level is below about 500 ng/dL. If the patient's TT level is at or above about 500 ng/dL this is an indication that the patient's testosterone levels are not the source of the patient's presenting symptoms. With 6| D:

findings of TT levels at or above about 500 ng/dL, the clinician should determine other reasons/causes for the patient's symptomatology. For example, the clinician may want to check the patient's thyroid stimulating hormone (TSH) levels to determine whether there are indications of thyroid problems, or perform a complete blood count (CBC) test to determine if the patient has indications of other disorders such as infection, anemia, immune disease, etc.

If the patient's initial TT levels are found to be below about 500 ng/dL, in the method of the preferred embodiment, the patient is started on about 140 mg testosterone cypionate (“TC”) injections about q7 (about every 7 days). Testosterone cypionate is an injectable synthetic androstane steroid derivative of testosterone.

About one week (about seven days) after the last of the first four consecutive weekly injections (the initial TC injection and three additional consecutive TC injections), hereafter the “one-month visit”, the next step in the method of the preferred embodiment is to test the patient's total testosterone and estradiol levels and conduct a CBC test. The CBC test also comprises the hemoglobin level.

As described in more detail below if indicated, these series of steps are performed at the one-month visit and, as indicated and appropriate, at the time of any subsequent and routine follow-up visits.

The next step in the method of the preferred embodiment is to determine whether the patient has elevated estrogen levels. If it is determined by the clinician that the patient has elevated estrogen levels, the patient is started on about 1 mg anastrozole, by mouth, about once weekly. In the preferred embodiment, the anastrozole, if indicated, is generally taken about 72 hours after each TC injection.

Although the aromatase inhibitor of the preferred embodiment is anastrozole, in other embodiments, other aromatase inhibitors or other agents may be used to control the patient's estrogen levels. For example, in other embodiments of the method, letrozole, exemestane, or other agents may be used in appropriate doses known in the art to reduce estrogen levels. In such embodiments, the letrozole, exemestane, or other agents are dosed in an amount that will prevent elevated estrogen levels as described in this disclosure.

After the step that includes determining the patient's TT level, the next step is to determine whether the patient's total testosterone level is in the therapeutic/stabilized range and whether the patient's presenting symptoms have improved. Certain actions are taken depending on these determinations. If the total testosterone levels are determined to be between about 605-1051 ng/dL, the patient is generally considered to be in the “therapeutic/stabilized range” for TT levels. If the patient is determined to be in this therapeutic/stabilized range for TT levels and the patient's presenting symptoms have improved, the patient is continued on the original prescribed TC dosing. If the TT levels are within the therapeutic/stabilized range but the presenting symptoms have not improved, the clinician then considers other sources/causes of the patient's presenting symptoms. If the TT is above the therapeutic/stabilized range (above about 1051 ng/dL), the TC dosage is adjusted downward about 20 mg (for example to about 120 mg weekly injections).

If the TT is determined to be below the therapeutic/stabilized range (below about 605 ng/dL), the TC dosage is adjusted upward about 20 mg (for example to about 160 mg weekly injections). When the total testosterone levels remain within the therapeutic/stabilized range for extended periods of time, for example, over several weeks, months, or years, the patient's total testosterone levels are said to be in a “steady state”.

After the step that includes determining the patient's hemoglobin level, the clinician determines whether the hemoglobin level is elevated. In the preferred embodiment, the patient's hemoglobin level is considered elevated if it exceeds 16 grams per deciliter (g/dL) or equivalent. If the patient's hemoglobin is determined to be elevated, the patient is started on about 81 mg of aspirin, daily, by mouth.

At the time of any subsequent follow-up visit, the clinician determines whether the Patient is Stable. Stability cannot be determined at the time of the one-month visit. However, in any visit after the patient has been taking testosterone for about at least three months, a determination of Stability can be potentially made.

Throughout the time period during which the patient is taking anastrozole, the clinician monitors the patient for symptoms of low estrogen levels (for example, hot flashes and/or new joint pains), and if there are no such low estrogen symptoms, maintain the patient on about the same dose of anastrozole. In the event the patient has symptoms of low estrogen levels, the anastrozole dose is decreased to about 0.5 mg, about once weekly by mouth. The timing of the anastrozole dosing may be changed depending upon whether bloating is observed before day three following administration of one of the TC injections. In such cases, the anastrozole is then taken about 48 hours after each TC injection. If the patient has bloating throughout the week following a TC injection, the anastrozole dose is increased by about 0.5 mg to be taken on the same day as the TC injection. This monitoring can be done weekly, monthly, or quarterly, or some other interval of time, depending on the clinical indications.

Throughout the time period during which the patient is taking TC, the clinician monitors the patient's hemoglobin levels. If the patient's hemoglobin level exceeds 16 g/dL and the patient is not already taking aspirin, the patient is started on about 81 mg of aspirin, daily, by mouth. If on follow-up, the patient's hemoglobin level exceeds 17 g/dL and the patient is already taking aspirin, blood is drawn from the patient, and/or the patient donates blood, in amounts sufficient to reduce the hemoglobin level to below 17 g/dL. This monitoring can be done weekly, monthly, or quarterly, or some other interval of time, depending on the clinical indications.

In the weeks, months, and years following the one-month visit and for so long as the patient is undergoing testosterone replacement therapy according to the method of the present disclosure, the clinician continues to follow the patient with subsequent follow-up visits. As used herein, the term “subsequent follow-up visit(s)” refers to any visit(s) after the one-month visit. The timing of these subsequent follow-up visits varies depending on the patient's response to therapy. Preferably, if the patient is determined to be otherwise Stable at the time of the one-month visit (or any subsequent follow-up visit), such that the patient would otherwise be determined to be Stable except for the Stability requirement that the patient's testosterone dosage amounts and intervals have remained consistent for about at least three months, the patient is seen again in a subsequent follow-up visit in about three months. If at the time of the one-month visit, the patient's presenting symptoms have not resolved or it is determined that the patient has symptoms of polycythemia, the patient is preferably seen again about one-month later.

Patients meeting all the requirements of Stability in a subsequent follow-up visit are seen in routine follow-up visits about every 3-6 months thereafter. As used herein, the term “routine follow-up visit” means any visit immediately following a visit in which the patient is determined to be Stable.

In other preferred embodiments, a method of treating hypogonadism in a human male patient, comprises the steps of administering to the patient determined to be in need of such treatment a therapeutically effective amount of a testosterone androstane steroid derivative (TASD) sufficient to maintain a total serum testosterone level within about a therapeutic/stabilized range.

In certain embodiments of this method, the TASD is testosterone cypionate.

In certain embodiments of this method, the method further comprises the step of administering a therapeutically effective amount of an aromatase inhibitor sufficient to maintain an estradiol level at or below 35 μg/mL or if the patient is determined to have an elevated estradiol level.

In certain embodiments of this method, if after administering the therapeutically effective amount of the aromatase inhibitor sufficient to maintain an estradiol level at or below 35 μg/mL the patient is determined to have an estrogen level below a predetermined level, the method further comprises the step of administering a revised therapeutically effective amount of the aromatase inhibitor sufficient to maintain the estrogen level above the predetermined level and the estradiol level at or below 35 μg/mL.

In certain embodiments of this method, the aromatase inhibitor is anastrozole.

In certain embodiments of this method, the method further comprises the step of administering a therapeutically effective amount of aspirin if the patient is determined to have an elevated hemoglobin level.

In certain embodiments of this method, the hemoglobin level is determined to be elevated if the hemoglobin level is greater than about 16 g/dL.

In certain embodiments of this method, the therapeutic/stabilized range is between about 605-1051 ng/dL.

In certain embodiments of this method, the therapeutic/stabilized range is between 605-1051 ng/dL.

In certain embodiments of this method, Stability occurs when the therapeutic/stabilized range is between 605-1051 ng/dL and the E2 is between 7-35 pg/mL.

In another preferred method, the method comprises the steps of determining a first total serum testosterone level of the patient; if the patient's first total serum testosterone level is below about 500 ng/dL, starting the patient on an initial dosage amount of about 140 mg of a testosterone androstane steroid derivative (TASD), administered about every 7 days; after four consecutive TASD administrations, determining a second total serum testosterone level, an estrogen level, and a hemoglobin level of the patient; if after four consecutive TASD administrations, the estrogen level is elevated, starting the patient on about 1 mg anastrozole, administered about once weekly; if after taking the 1 mg anastrozole administered about once weekly the patient has a low estrogen level, decreasing the anastrozole dosage to about 0.5 mg, administered about once weekly: if after the four consecutive TASD administrations the patient has an elevated hemoglobin level, starting the patient on aspirin, administered daily; if after the four consecutive TASD administrations, the second total serum testosterone level is within a therapeutic/stabilized range and the four consecutive TASD administrations are therapeutically effective, continuing the patient on the initial dosage amount of the TASD; if after the four consecutive TASD administrations, the second total serum testosterone level is above the therapeutic/stabilized range, decreasing the initial dosage amount of TASD by about 20 mg; if after the four consecutive TASD administrations, the second total serum testosterone level is below the therapeutic/stabilized range, increasing the initial dosage amount of TASD by about 20 mg.

In certain embodiments of this method, the testosterone androstane steroid derivative is testosterone cypionate.

In certain embodiments of this method, the therapeutic/stabilized range is about 605-1051 ng/dL.

In certain embodiments of this method, the estrogen level is elevated if an estradiol level is greater than about 35 μg/mL.

In certain embodiments of this method, a determination is made that the estrogen level is elevated based on clinical indications.

In certain embodiments of this method, the hemoglobin level is elevated if the hemoglobin level is greater than 16 g/dL.

In certain embodiments of this method, the method further comprises the step of determining subsequent total serum testosterone levels, and, using such subsequent total serum testosterone levels, administering the TASD in revised dosage amounts sufficient to maintain a steady state total serum testosterone level within about the therapeutic/stabilized range.

In certain embodiments of this method, the therapeutic/stabilized range is about 605-1051 ng/dL.

In certain embodiments of this method, the testosterone androstane steroid derivative is testosterone cypionate.

In certain embodiments of this method, the method further comprises the step of determining whether the patient has reached Stability.

### BRIEF DESCRIPTION OF THE DRAWING

FIG. 1 is a flow chart of a method of treating hypogonadism in men, in accordance with a preferred embodiment.

### DETAILED DESCRIPTION OF THE PREFERRED EMBODIMENT

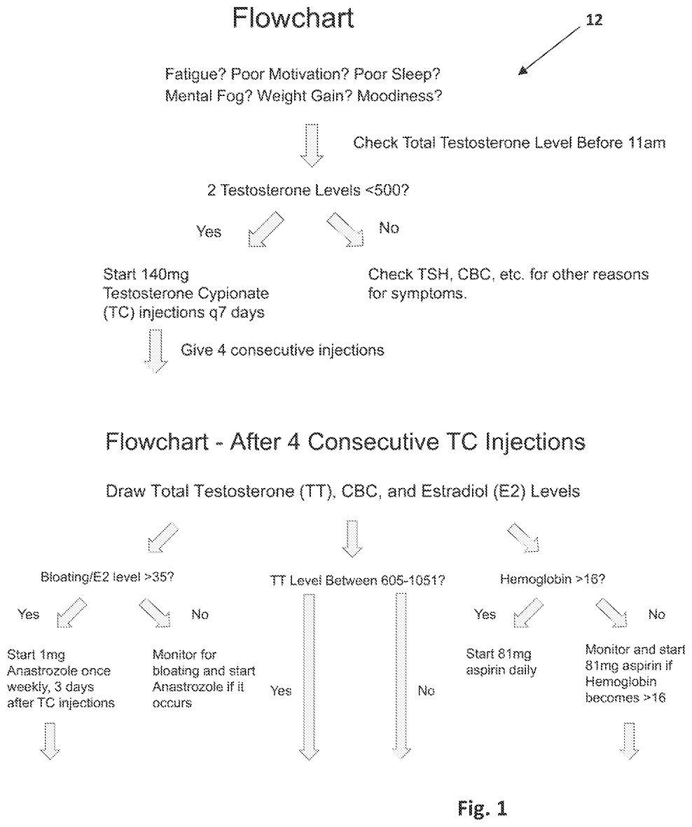

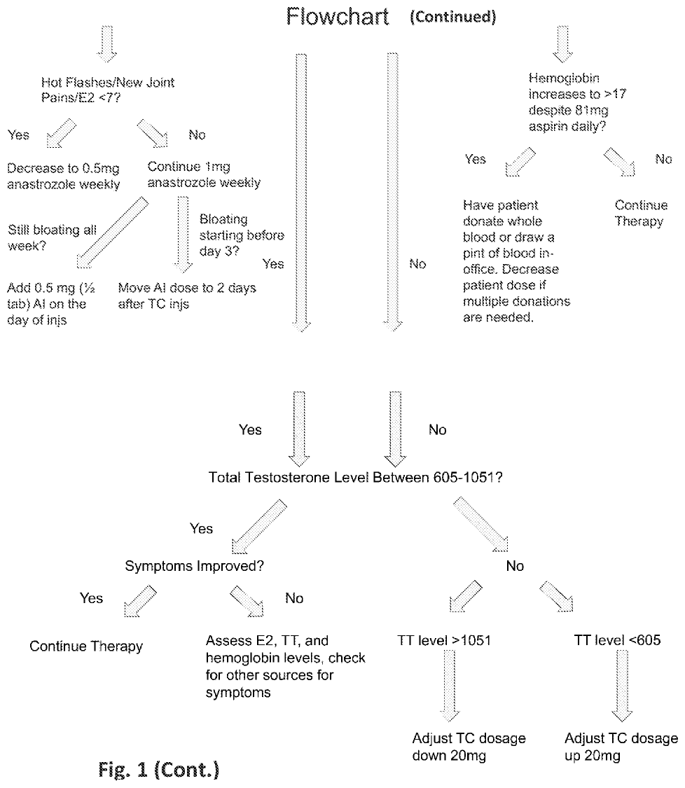

Referring to FIG. 1, there is shown the method for treating hypogonadism in men 12, in accordance with preferred embodiments. As used herein, the terms “a” or “an” shall mean one or more than one. The term “plurality” shall mean two or more than two. The term “another” is defined as a second or more. The terms “including” and/or “having” are open ended (e.g., comprising). The term “or” as used herein is to be interpreted as inclusive or meaning any one or any combination. Therefore, “A, B or C” means “any of the following: A; B; C; A and B; A and C; B and C; A, B and C”. An exception to this definition will occur only when a combination of elements, functions, steps or acts are in some way inherently mutually exclusive.

Reference throughout this document to “one embodiment,” “certain embodiments,” “an embodiment,” or similar term means that a particular feature, structure, or characteristic described in connection with the embodiment is included in at least one embodiment of the present disclosure. Thus, the appearances of such phrases in various places throughout this specification are not necessarily all referring to the same embodiment. Furthermore, the particular features, structures, steps, actions, or characteristics may be combined in any suitable manner in one or more embodiments without limitation. All publications, patents, and patent applications cited herein are hereby incorporated by reference in their entirety for all purposes to the extent not inconsistent to the present disclosures.

### General Summary of Preferred Embodiment

Referring to the figure, and as will be discussed in more detail below, the method for treating hypogonadism in men 12 of the preferred embodiment generally comprises the steps of: determining whether a patient has presenting symptoms consistent with low testosterone; determining a total testosterone level (“TT”) of the patient; if the TT level is below about 500 ng/dL, administering to the patient about 140 mg Testosterone Cypionate (“TC”) injections about q7 (about every 7 days); about one week after the first four consecutive testosterone replacement injections (after four consecutive TC injections), determining the patient's TT, CBC (complete blood count), and estradiol (E2) levels; determining whether the patient has symptoms consistent with elevated estrogen levels, (if the patient has bloating, water weight gain, or E2 levels above about 35 μg/mL), starting the patient on about 1 mg anastrozole, once weekly by mouth, and monitoring the patient for complaints of low estrogen levels (hot flashes and/or new joint pains), and if there are no such symptoms, continuing the patient with about the same dose of anastrozole, and if there are such symptoms consistent with low estrogen levels, decreasing the anastrozole dosage to about 0.5 mg, once weekly; if the TT is between about 605-1051 ng/dL (“therapeutic/stabilized range”) and the presenting symptoms have improved, continuing the patient on about the 140 mg TC therapy; if the TT is between about 605-1051 ng/dL but the presenting symptoms have not improved, assessing the E2, TT, and hemoglobin levels and considering other sources and/or causes of the symptoms; if the TT is above about 1051 ng/dL, adjusting the TC dosage downward about 20 mg; if less than about 605 ng/dL, adjusting the TC dosage upward about 20 mg; continuing to monitor the patient periodically, for example, about once every six to twelve months, and repeating the steps listed above beginning with “about one month after the first injection . . . ”.

### DETAILED DESCRIPTION

The method for treating hypogonadism in men 12 of preferred embodiments is used to achieve “Stabilization”. Except as otherwise provided herein, the terms “Stable”, “Stability”, “Stabilized” and “Stabilization” are defined in this disclosure as “the point at which the patient's presenting symptoms have resolved, polycythemia, if any, has stopped, and testosterone dosage amounts and intervals remain consistent for at least three months.”

As used herein, the term “about” refers to a value that is +/−10% of a recited value. For example, a dose of about 140 mg TC refers to a dose that contains from 126 mg to 154 mg of TC. When referring to days, the term “about” refers to a value of +/−1 day. For example, an event (e.g., a TT measurement) that occurs on about day seven or about one week may occur from day six to day eight.

Unless stated otherwise, when a range is provided in this disclosure, the range should be considered inclusive, such that the beginning and ending values are included in the referenced range.

As used herein, the term “consistent” when referring to testosterone dosage amounts and/or intervals means the dosage amounts have been at about the same level and the intervals have been about the same.

Although for ease of understanding, the methods of the preferred embodiments are sometimes set forth in numbered steps, those skilled in the art will recognize that in certain of the embodiments, not all the steps need necessarily be performed. Likewise, not all the actions within a certain step need necessarily be performed. Rather, the steps set forth below illustrate by way of example, not by way of limitation, the principles of the methods and this invention. The description will clearly enable one skilled in the art to practice the methods of the invention, including what is presently believed to be the best mode of carrying out the invention. This disclosure is not to be limited in any way, but rather includes all that falls within the scope and spirit of this disclosure and the accompanying claims.

Step 1: Determining whether the patient has symptoms consistent with low testosterone.

In the preferred embodiment, the first step in the method for treating hypogonadism in men 12 begins with determining whether the patient has presenting symptoms consistent with low testosterone. In this disclosure, the word “symptoms” refers to subjective reports from the patient as well as those that can be measured or observed by the clinician. Unless otherwise indicated, the term “presenting symptoms” means the symptoms present at the time of an initial visit. In preferred embodiments, the symptoms that are consistent with low testosterone include fatigue, poor motivation, decreased libido, poor sleep quality, weight gain, mental fogginess (lack of mental clarity), moodiness, depression, loss of muscle mass, and erectile dysfunction. Most of these symptoms are self-reported by the patient. Others, for example, weight gain, poor sleep quality, and loss of muscle mass can be either self-reported, observed, or objectively measured.

Clinicians commonly make assessments and determinations about whether the patient has low testosterone symptoms based upon the patient's responses or behavior during patient interviews. For example, the patient may display a flat affect or report being irritable or unhappy. Thus, the clinician uses a combination of information and test results, along with the clinician's experience and expertise, in making determinations about the patient's condition, including, but not limited to whether the patient has symptoms consistent with low testosterone.

In this disclosure, unless otherwise provided, the term “clinician(s)” refers to the healthcare professional responsible for administering and/or monitoring the testosterone replacement therapy of the present disclosure. The clinician, therefore, for example, can be a physician, a physician assistant (PA), nurse, or other professional qualified by licensing, experience, and training to administer and monitor testosterone replacement therapy. As testosterone replacement therapy takes place over an extended period of time, the word clinician does not necessarily refer to the same clinician. Rather, multiple clinicians will likely play a role in administering and monitoring the patient's testosterone replacement therapy over the months and years of administering testosterone replacement therapy in accordance with the methods of this disclosure.

Step 2: Determining the Patient's total testosterone level.

If the patient has symptoms consistent with low testosterone, the next step is to determine the patient's total testosterone level. In this disclosure, the terms “total testosterone level” and “total serum testosterone level” refer to the total amount of testosterone present in a sample of the patient's blood (serum). The total amount of testosterone includes both blood borne testosterone that is attached to proteins and “free testosterone” that is not attached to proteins. In preferred embodiments, the patient's TT level is determined by use of a conventional and commercially available total serum testosterone test conducted in a conventional manner. For example, a needle is used to draw blood from a vein in the patient's arm or hand. Preferably, the test is conducted in the morning before about 11 a.m., when testosterone levels tend to be highest. However, the test need not be performed in the morning. It is also preferred that the TT level be measured while the patient is fasting (i.e., through fasting blood tests). Thus, it is preferred that the patient not have consumed any food or liquids (except water) for several hours prior to the blood being drawn for any of the tests called upon in executing the methods of this disclosure.

Although in the preferred embodiment, the patient's total testosterone is measured through a total testosterone test, in other embodiments, other tests may be used to determine the patient's level of circulating testosterone and Stability. For example, the patient's level of free testosterone can be measured through a free testosterone test. Alternatively, a bioavailable testosterone test can be performed to measure free testosterone and testosterone that's loosely attached to albumin in the blood. In other embodiments, an SHBG test can be performed that measures the amount of circulating testosterone bound to sex hormone binding globulin (SHBG). In most cases, using any of the three tests will provide meaningful information concerning whether the patient is suffering from low testosterone levels. In cases in which the total testosterone level is believed to not adequately reflect biological activity, free or bioavailable testosterone can be measured, and estimates/conversions can be made concerning the total testosterone level and dosing requirements needed to obtain Stability. It is preferable that the respective methods employed to measure the initial total testosterone and other levels be consistent with the respective methods used to monitor and re-measure total testosterone and other levels prior to and during testosterone therapy. In the event that different methods are used to measure respective testosterone and other values, it is desirable that the respective measured values be adjusted, by, for example, using a correlation factor or other technique, tool, scale, and/or conversion methodology known in the art so that meaningful clinical decisions, comparisons, and/or necessary dosing adjustments can be made.

The results of the total testosterone test of the preferred embodiment are preferably expressed in terms of nanograms per deciliter (ng/dL). Although this disclosure provides a method for treating hypogonadism in men using ng/dL units, the units can be expressed in other equivalent units well known in the art. For example, the units can be expressed in nanomoles per liter (nmol/L).

In other embodiments of the method, additional blood work is performed at the time the initial total testosterone levels are measured. In such embodiments, the testing comprises one or more tests to determine the patient's estradiol (E2) level, thyroid stimulating hormone (TSH) level, prostate-specific antigen (PSA) level, CBC, A1c, and/or vitamin D level. These additional studies may be used by the clinician, for example, to determine whether any of the patient's presenting symptoms result from other conditions.

Step 3: If the patient's TT level is below about 500 ng/dL, starting the patient on 140 mg Testosterone Cypionate (“TC”) injections q7 (every 7 days)

After the step of measuring the patient's total testosterone level, the next step of the method of the preferred embodiment is to start the patient on about 140 mg testosterone cypionate (“TC”) injections about q7 (about every 7 days) if the patient's TT level is below about 500 ng/dL. If the patient's TT level is at or above about 500 ng/dL this is an indication that the patient's testosterone levels are not the source of the patient's presenting symptoms. With findings of TT levels at or above about 500 ng/dL, the clinician should determine other reasons/causes for the patient's symptomatology. For example, the clinician may want to check the patient's thyroid stimulating hormone (TSH) levels to determine whether there are indications of thyroid problems, or perform a complete blood count (CBC) test to determine if the patient has indications of other disorders such as infection, anemia, immune disease, etc.

If the patient's initial TT levels are found to be below about 500 ng/dL, in the method of the preferred embodiment, the patient is started on about 140 mg testosterone cypionate (“TC”) injections about q7 (about every 7 days). Testosterone cypionate is an injectable synthetic androstane steroid derivative of testosterone.

Although the testosterone cypionate injectable route of administration is used in the method of the preferred embodiment, in other embodiments, other forms of testosterone replacement therapeutics may be used. For example, in other embodiments, oral pills, creams, pellets, patches, or other injectables well known in the art may be used. For example, testosterone enanthate may be used. Appropriate dosage changes and administration rates may be made in such alternative forms of testosterone replacement therapeutics so as to maintain Stability as provided in this disclosure.

Step 4a: Determining the patient's TT, CBC (including hemoglobin level), and estradiol (E2) Levels.

About one week (about seven days) after the last of the first four consecutive weekly injections (the initial TC injection and three additional consecutive TC injections), hereafter the “one-month visit”, the next step in the method of the preferred embodiment is to test the patient's total testosterone and estradiol levels and conduct a CBC test. The CBC test also comprises the hemoglobin level. As mentioned, these tests are preferably performed, respectively, in the same manner and under the same conditions as the respective previous tests.

As described in more detail below in Step 7, if indicated, Steps 4a-4e are performed at the one-month visit and Steps 4a-4f are performed at the time of any subsequent and routine follow-up visits.

Step 4b: Determine whether the patient has elevated estrogen levels

The next step in the method of the preferred embodiment is to determine whether the patient has elevated estrogen levels. In the method of the preferred embodiment, this determination is based on the patient's symptomology and/or the results of blood tests. For example, bloating and water weight gain are physical symptoms that are evidence of elevated estrogen levels. Estradiol (E2) levels above about 35 μg/mL is also evidence of elevated estrogen levels.

Step 4c: If the patient has elevated estrogen levels, start the patient on anastrozole

If it is determined by the clinician that the patient has elevated estrogen levels, the patient is started on about 1 mg anastrozole, by mouth, about once weekly. Anastrozole is an aromatase inhibitor which helps prevent the conversion of TC to estradiol when taken between about 48-72 hours post-TC injection. As the half-life of anastrozole in humans is about 30-60 hours, the timing of the anastrozole doses is important for maintaining consistent TT levels. Therefore, in the preferred embodiment, the anastrozole, if indicated, is generally administered about 72 hours after each TC injection.

Although the aromatase inhibitor of the preferred embodiment is anastrozole, in other embodiments, other aromatase inhibitors or other agents may be used to control the patient's estrogen levels. For example, in other embodiments of the method, letrozole, exemestane, or other agents may be administered in appropriate doses known in the art to reduce estrogen levels. In such embodiments, the letrozole, exemestane, or other agents are dosed and administered in an amount that will prevent elevated estrogen levels as described in this disclosure.

Step 4d: Determine whether the TT level is in the therapeutic/stabilized range and whether the patient's presenting symptoms have improved and take certain actions depending on these determinations

At this visit, after the step that includes determining the patient's TT level, the next step is to determine whether the patient's total testosterone level is in the therapeutic/stabilized range and whether the patient's presenting symptoms have improved. Certain actions are taken depending on these determinations. In the method of preferred embodiments, whether a patient's complaints, signs, symptoms, conditions, and the like have improved or resolved is a clinical determination made by the clinician. The clinician makes a determination whether any complaints, signs, symptoms, conditions, and the like have improved or resolved and whether the patient has therapeutically benefitted from any therapy, modality, treatment, medication, etc. Thus, the clinician clinically determines whether such therapy, modality, treatment, medication, etc. has been therapeutically effective. In making these and other clinical determinations, the clinician uses a combination of information and test results, along with the clinician's experience, expertise, and judgment in making the determination that the patient's total testosterone level is in the therapeutic/stabilized range and whether the patient's presenting symptoms have improved.

In this step, if the total testosterone levels are determined to be between about 605-1051 ng/dL the patient is generally considered to be in the “therapeutic/stabilized range” for TT levels. If the patient is determined to be in this therapeutic/stabilized range for TT levels and the patient's presenting symptoms have improved, the patient is continued on the original prescribed TC dosing.

If the TT levels are within the therapeutic/stabilized range but the presenting symptoms have not improved, the clinician then considers other sources/causes of the patient's presenting symptoms. For example, the clinician may assess the E2, TT, and hemoglobin levels in determining other potential sources/causes of the presenting symptoms.

If the TT is above the therapeutic/stabilized range (above about 1051 ng/dL), the TC dosage is adjusted downward about 20 mg (for example to about 120 mg weekly injections).

If the TT is determined to be below the therapeutic/stabilized range (below about 605 ng/dL), the TC dosage is adjusted upward about 20 mg (for example to about 160 mg weekly injections).

When the total testosterone levels remain within the therapeutic/stabilized range for extended periods of time, for example, over several weeks, months, or years, the patient's total testosterone levels are said to be in a “steady state”.

Step 4e: Determine whether the Patient's Hemoglobin is elevated and take certain actions depending on this determination

After the step that includes determining the patient's hemoglobin level, the clinician determines whether the hemoglobin level is elevated. In the preferred embodiment, the patient's hemoglobin level is considered elevated if it exceeds about 16 grams per deciliter (g/dL) or equivalent. If the patient's hemoglobin is determined to be elevated, the patient is started on about 81 mg of aspirin, daily, by mouth.

Step 4f: Determine whether the Patient is Stable

At the time of any subsequent follow-up visit, the clinician determines whether the Patient is Stable. Because the determination of Stability requires that the “testosterone dosage amounts and intervals remain consistent for about at least three months”, a determination of Stability can only be made after the patient has been administered testosterone for about at least three months. Thus, Stability cannot be determined at the time of the one-month visit. However, in any visit after the patient has been taking testosterone for about at least three months, a determination of Stability can be potentially made.

Step 5: Continue to Monitor the patient for symptoms of low estrogen levels and take certain actions depending on the patient's symptoms

Throughout the time period during which the patient is taking anastrozole, the clinician monitors the patient for symptoms of low estrogen levels (for example, hot flashes and/or new joint pains), and if there are no such low estrogen symptoms, maintain the patient on about the same dose of anastrozole. In the event the patient has symptoms of low estrogen levels, the anastrozole dose is decreased to about 0.5 mg, about once weekly by mouth. The timing of the anastrozole dosing may be changed depending upon whether bloating is observed before day three following administration of one of the TC injections. In such cases, the anastrozole is then taken about 48 hours after each TC injection. If the patient has bloating throughout the week following a TC injection, the anastrozole dose is increased by about 0.5 mg to be taken on the same day as the TC injection. This monitoring can be done weekly, monthly, or quarterly, or some other interval of time, depending on the clinical indications.

Step 6: Continue to Monitor the patient's hemoglobin levels and take certain actions depending on these levels

Throughout the time period during which the patient is taking TC, the clinician monitors the patient's hemoglobin levels. If the patient's hemoglobin level exceeds 16 g/dl and the patient is not already taking aspirin, the patient is started on about 81 mg of aspirin, daily, by mouth. If on follow-up, the patient's hemoglobin level exceeds 17 g/dL and the patient is already taking aspirin, blood is drawn from the patient, and/or the patient donates blood, in amounts sufficient to reduce the hemoglobin level to below 17 g/dL. This monitoring can be done weekly, monthly, or quarterly, or some other interval of time, depending on the clinical indications.

Step 7: Repeat Steps 4-6 for so long as the patient is undergoing TRT.

In the weeks, months, and years following the one-month visit and for so long as the patient is undergoing testosterone replacement therapy according to the method of the present disclosure, the clinician continues to follow the patient with subsequent follow-up visits. As used herein, the term “subsequent follow-up visit(s)” refers to any visit(s) after the one-month visit. The timing of these subsequent follow-up visits varies depending on the patient's response to therapy. Preferably, if the patient is determined to be otherwise Stable at the time of the one-month visit (or any subsequent follow-up visit), such that the patient would otherwise be determined to be Stable except for the Stability requirement that the patient's testosterone dosage amounts and intervals have remained consistent for about at least three months, the patient is seen again in a subsequent follow-up visit in about three months. If at the time of the one-month visit, the patient's presenting symptoms have not resolved or it is determined that the patient has symptoms of polycythemia, the patient is preferably seen again about one-month later.

Patients meeting all the requirements of Stability in a subsequent follow-up visit are seen in routine follow-up visits about every 3-6 months thereafter. As used herein, the term “routine follow-up visit” means any visit immediately following a visit in which the patient is determined to be Stable.

At the time of any subsequent follow-up visit, the Steps 4-6 are repeated.

In other preferred embodiments, a method of treating hypogonadism in a human male patient, comprises the steps of: administering to a patient determined to be in need of such treatment a therapeutically effective amount of a testosterone androstane steroid derivative (TASD) sufficient to maintain a total serum testosterone level within about a therapeutic/stabilized range.

In certain embodiments of this method, the TASD is testosterone cypionate.

In certain embodiments of this method, the method further comprises the step of administering a therapeutically effective amount of an aromatase inhibitor sufficient to maintain an estradiol level at or below 35 μg/mL, if the patient is determined to have an elevated estrogen level.

In certain embodiments of this method, if after administering the therapeutically effective amount of the aromatase inhibitor sufficient to maintain an estradiol level at or below 35 μg/mL, the patient is determined to have an estrogen level below a predetermined level, the method further comprises the step of administering a revised therapeutically effective amount of the aromatase inhibitor sufficient to maintain the estrogen level above the predetermined level and the estradiol level at or below 35 μg/mL.

In certain embodiments of this method, the aromatase inhibitor is anastrozole. In certain embodiments of this method, the method further comprises the step of administering a therapeutically effective amount of aspirin, if the patient is determined to have an elevated hemoglobin level.

In certain embodiments of this method, the hemoglobin level is determined to be elevated if the hemoglobin level is greater than about 16 g/dL.

In certain embodiments of this method, the serum total testosterone level therapeutic/stabilized range is between about 605-1051 ng/dL.

In certain embodiments of this method, the therapeutic/stabilized range is between 605-1051 ng/dL.

In certain embodiments of this method, Stability occurs when the therapeutic/stabilized range is between 605-1051 ng/dL and the E2 is between 7-35 pg/mL.

In another preferred method, the method comprises the steps of, determining a first total serum testosterone level of the patient; if the patient's first total serum testosterone level is below about 500 ng/dL, starting the patient on an initial dosage amount of about 140 mg of a testosterone androstane steroid derivative (TASD), administered about every 7 days; after four consecutive TASD administrations, determining a second total serum testosterone level, an estrogen level, and a hemoglobin level of the patient; if after four consecutive TASD administrations, the estrogen level is elevated, starting the patient on about 1 mg anastrozole, administered about once weekly; if after taking the 1 mg anastrozole administered about once weekly the patient has a low estrogen level, decreasing the anastrozole dosage to about 0.5 mg, administered about once weekly; if after the four consecutive TASD administrations the patient has an elevated hemoglobin level, starting the patient on aspirin, administered daily; if after the four consecutive TASD administrations, the second total serum testosterone level is within a therapeutic/stabilized range and the four consecutive TASD administrations are therapeutically effective, continuing the patient on the initial dosage amount of the TASD; if after the four consecutive TASD administrations, the second total serum testosterone level is above the therapeutic/stabilized range, decreasing the initial dosage amount of TASD by about 20 mg; if after the four consecutive TASD administrations, the second total serum testosterone level is below the therapeutic/stabilized range, increasing the initial dosage amount of TASD by about 20 mg.

In certain embodiments of this method, the testosterone androstane steroid derivative is testosterone cypionate.

In certain embodiments of this method, the therapeutic/stabilized range is about 605-1051 ng/dL.

In certain embodiments of this method, the estrogen level is elevated if an estradiol level is greater than about 35 μg/mL.

In certain embodiments of this method, the estrogen level is low if an estradiol level is less than about 7 μg/mL.

In certain embodiments of this method, the therapeutic/stabilized range is between 605-1051 ng/dL.

In certain embodiments of this method, Stability occurs when the therapeutic/stabilized range is between 605-1051 ng/dL and the E2 is between 7-35 pg/mL.

In certain embodiments of this method, a determination is made that the estrogen level is elevated based on clinical indications.

In certain embodiments of this method, the hemoglobin level is elevated if the hemoglobin level is greater than about 16 g/dL.

In certain embodiments of this method, the method further comprises the step of determining subsequent total serum testosterone levels, and, using such subsequent total serum testosterone levels, administering the TASD in revised dosage amounts sufficient to maintain a steady state total serum testosterone level within about the therapeutic/stabilized range.

In certain embodiments of this method, the therapeutic/stabilized range is about 605-1051 ng/dL.

In certain embodiments of this method, the therapeutic/stabilized range is between 605-1051 ng/dL.

In certain embodiments of this method, Stability occurs when the therapeutic/stabilized range is between 605-1051 ng/dL and the E2 is between 7-35 pg/mL.

In certain embodiments of this method, the testosterone androstane steroid derivative is testosterone cypionate.

In certain embodiments of this method, the method further comprises the step of determining whether the patient has reached Stability.

### Example

The following example is offered to illustrate, but not limit the claimed invention.

Study: A study (“Study”) was conducted to determine an appropriate testosterone cypionate (TC) dosing that eliminates side effects, including, but not limited to, the polycythemia effect seen in patients with excessive TC administration. Patient hemoglobin and total testosterone (TT) levels were used in this Study to determine when a patient becomes “stable” on treatment. The terms “stable”, “stability”, “stabilized” and “stabilization” are defined in this Study as “the point at which a patient's symptoms have resolved, polycythemia has stopped, and TC dosage has remained consistent for at least 3 months.” Polycythemia associated with testosterone replacement therapy (TRT) has commonly been controlled through frequent patient blood donation. However, this Study shows that it is possible to idealize TC dosing and minimize or eliminate side effects, including polycythemia. Several TT “normal” ranges can be found in the literature. For example, the Endocrine Society identifies the normal range for TT levels in males to be 264-916 ng/dL (1,17). However, LabCorp Diagnostics has used the previously accepted “normal” range of 348-1197 ng/dL until 2017 and Quest Diagnostics uses a reference range of 250-1110 ng/dL.

This Study focuses on testosterone cypionate injections. The injectable route of administration has been found to be the most consistent and easiest to control over time in terms of serum testosterone levels due to the well-known half-life and elimination time of the medication. TC has a half-life of 7-8 days and is eliminated from the body in approximately 30 days when injected intramuscularly. The injections can be administered once weekly, and it takes 1 month for testosterone levels to reach steady state concentrations in the body. This is very important due to the side effects that are seen when testosterone levels fluctuate in the body. Side effects include, but are not limited to, acne, elevating estradiol levels (due to conversion of TC to estradiol), polycythemia, hair loss, and aggression.

Patients' responses to TC injections administered every 7 days were tracked and the resulting effects of TC dosing on cholesterol, A1c, and PSA levels were documented once stable TT levels were reached.

Objectives: The objectives of the Study were to identify an appropriate level of TC dosing in males that maximizes benefits to the patient while minimizing side effects such as polycythemia; to offer an alternative “normal range” for testosterone levels in males once stabilized and to establish a starting dosage with appropriate dosing range. And finally, another objective was to document the effects of testosterone cypionate dosing on total cholesterol, A1c, and PSA once TT levels within the therapeutic/stabilized range have been reached.

Subjects: Two cohorts were studied. The two cohorts studied were males that had previously been on TRT and males who had never been on any form of TRT. Blood work was collected from all patients at their initial visit, after 1 month of treatment, after 3 months of treatment, and then at 6 months, 9 months, 12 months, etc. as patients continued treatment. Data was collected for each cohort including the length of time that it took to stabilize on treatment and the average dosage injected weekly once patients had reached stabilization. Age, weight, and height were also tracked in this Study, but did not have an effect on the results of treatment.

Summary of Results: In this Study, it was found that patients stabilized when total testosterone levels reached a range between 605-1051 ng/dL. The effects of testosterone cypionate dosing at stable levels were tracked and discussed for total cholesterol, PSA, and A1c. Fasting blood work (including TT, estradiol, PSA, CBC, CMP, A1c, and vitamin D levels) was collected from patients at their first visit. Subsequently, the patients' PSA and hemoglobin A1c levels were consistently tracked from their blood work until they reached stability, such that they no longer needed to donate blood or change their dose of TC.

The primary negative side effect with conventional TRT is secondary polycythemia. This has been a significant source of concern for most providers and has caused providers to shy away from treatment for fear of putting the patient at risk for clotting. However, data resulting from this Study indicates that most of the patients having issues with polycythemia were either being overdosed or the frequency of the dosing was incorrect. The novel and precise timing of the injections utilized in this Study, as well as use of an estrogen blocker (anastrozole) were found to be vital in helping the patient avoid continued issues with polycythemia. The data from this study indicates that certain timing of the administration of TC injections along with certain dosing of anastrozole decreases the overall amount of fluctuation of both testosterone and estradiol levels in the blood. Consistency of both their TT and estradiol levels needed to be reached before the patients were able to reach a point of stabilization with their treatment.

In previous trials, patients were either treated with daily topical testosterone or testosterone cypionate injections with dosing regimens ranging from 50 mg weekly to 300 mg every other week. However, in this Study TC injections were administered every 7 days, and blood samples were collected at 7 days post-TC injection and after at least 4 consecutive weeks of injections. These parameters were set based on the half-life (7-8 days) and elimination time (approx. 1 month) of testosterone cypionate. Collecting blood samples at the 4-week mark provides more consistent, accurate, and useful information regarding the TT blood level than if the samples are collected earlier.

Blood samples were then sent to an outsourced laboratory, Lab Tech Diagnostics. Samples were drawn at the patient's initial visit, after their first 4 consecutive TC injections, after 3 months of treatment, and at 3-month intervals thereafter. Testosterone, estradiol, and hemoglobin levels were tracked with every blood sample. PSA and cholesterol levels were checked every 3 months. A1c levels were checked with initial blood work, and then once per year if A1c was within normal limits (WNL) or every 3 months if A1c was >5.6.

Given, the primary goal was to determine a stabilization point such that the patient achieved maximum benefit with minimal side effects (e.g., elimination of polycythemia) from the treatment, the challenge was to identify a TC dose at which the patient's hemoglobin stopped increasing. The Study indicates that hemoglobin levels are the most specific metric indicating the point at which the patient's treatment reaches stabilization. Patients were advised to start 81 mg aspirin daily if their hemoglobin started increasing after starting treatment. Adding aspirin as a minor blood thinner to the patient's treatment helped to either decrease hemoglobin levels or slow the rate of increase of hemoglobin levels. Dosage adjustments of TC were made in 20 mg (0.1 ml) increments. Assessment of new dosages were not made until after four consecutive injections at the new dose and at 7 days after their last injection. After analysis of data, there were notable similarities in the Total Testosterone, estradiol, and hemoglobin levels once patients reached stabilization on treatment. There were also differences seen between patients that had been on TRT in the past through other clinics and those that started treatment with our clinic. Patients who had previously been on TRT took longer to stabilize on therapy than those who had never been on TRT.

Results: Chief complaints/symptoms identified by the patients during their initial office visit are listed in Table 1 below. Note that all of these complaints/symptoms were reported by the patient to be well-controlled once stable on treatment.

TABLE 1

| Chief Complaint/Condition | Percent of |
|---|---|
| on Initial Visit | Patients |
| Fatigue | 98.3% |
| Poor motivation | 78.3% |
| Decreased libido | 45% |
| Poor sleep quality | 46.7% |
| Weight gain | 33.3% |
| Mental fogginess | 26.7% |
| Moodiness | 20% |
| Loss of muscle mass | 15% |
| Erectile dysfunction | 8.3% |

TABLE 2

| | Patients | | Patients who | |
|---|---|---|---|---|
| Event | previously | | have never | |
| (All data are averages) | on TRT | | been on TRT | |
| Starting TT dose | 167.3 | mg | 138.7 | mg |
| TC Dosage once stable | 147.0 | mg | 149.3 | mg |
| Starting TT level | 479.83 | ng/dl (21 pts) | 347.77 | ng/dl |
| TT at week 5 | 805.90 | ng/dl | 705.43 | ng/dl |
| TT once stable | 780.10 | ng/dl | 794.53 | ng/dl |
| Initial Hemoglobin level | 15.78 | g/dl | 15.06 | g/dl |
| Hemoglobin at 5 weeks | 16.22 | g/dl | 15.33 | g/dl |
| Hemoglobin at stabilization | 15.70 | g/dl | 15.79 | g/dl |
| Estradiol level at | 17.5 | pg/ml | 18.2 | pg/ml |
| stabilization | | | | |
| Anastrozole dose at | 0.90 | mg | 0.75 | mg |
| stabilization | | | | |
| Initial A1c | 5.66% | (24 pts) | 5.99% | (23 pts) |
| A1c at stabilization | 5.52% | (24 pts) | 5.60% | (23 pts) |
| Initial total | 207.93 | (28 pts) | 195.78 | (27 pts) |
| cholesterol level | | | | |
| Total cholesterol at | 195.86 | (28 pts) | 186.11 | (27 pts) |
| stabilization | | | | |
| Initial PSA level | 1.017 | ng/ml | 0.859 | ng/ml |

| | | |
|---|---|---|
| PSA level at stabilization | 1.163 ng/ml | 1.090 ng/ml |
| | (14.36% increase) | (26.89% increase) |

Cohort 1: Referring to Table 2, above, for patients who had previously been on TRT:
Average starting TT dose was 167.3 mg and average dosage once stable was 147 mg.
Average starting TT level was 479.83 ng/dL, avg. TT level at week 5 was 805.9 ng/dL, and avg. TT level once stable was 780.1 ng/dL.
Average initial hemoglobin level was 15.78 g/dl, avg. hemoglobin at week 5 was 16.22 g/dl, and avg. hemoglobin at stabilization was 15.70 g/dl.
Average estradiol level at stabilization was 17.5 pg/ml.
Average anastrozole dose at stabilization was 0.9 mg.
Average initial A1c was 5.66% and avg. A1c at stabilization was 5.52%.
Average initial total cholesterol level was 207.93 mg/dl and avg. total cholesterol at stabilization was 195.86 mg/dl.
Average initial PSA level was 1.017 ng/ml and avg. PSA level at stabilization was 1.163 ng/ml (14.36% increase).

Cohort 2: Referring again to Table 2 above, for patients who had never been on TRT:
Average starting TT dose was 138.7 mg and avg. dosage once stable was 149.3 mg.
Average starting TT level was 347.77 ng/dL, avg. TT level at week 5 was 705.43 ng/dL, and avg. TT level once stable was 794.53 ng/dL.
Average starting hemoglobin level was 15.06 g/dl, avg. hemoglobin at week 5 was 15.33 g/dl, and avg. hemoglobin at stabilization was 15.79 g/dl.
Average estradiol level at stabilization was 18.2 pg/ml.
Average anastrozole dose at stabilization was 0.75 mg.
Average initial A1c was 5.99% and avg. A1c at stabilization was 5.60%.
Average initial total cholesterol level was 195.78 mg/dl and avg. total cholesterol at stabilization was 186.11 mg/dl.
Average initial PSA level was 0.859 ng/ml and avg. PSA level at stabilization was 1.090 ng/ml (26.89% increase).

Analysis of Data: Analysis of the data revealed significant similarities in the TT, estradiol, and hemoglobin levels once they reached stabilization on treatment. Once a patient reached stability on treatment, they no longer needed to donate blood (polycythemia was eliminated) or change their TC dose going forward. There were also differences seen between patients that had been on TRT in the past and those that started treatment with our clinic. Patients who had previously been on TRT in any form took an average of 9.7 months longer to stabilize than patients who had never been on TRT.

Based on the data gathered during this study, the suggested starting testosterone cypionate dosage is 140 mg every 7 days. This was the average dosage being administered to patients at the point of stabilization. The TT level for all patients in the study stabilized between 605-1051 ng/dL Ninety percent (54 out of 60) of patients treated in this study needed to take an estrogen blocker (anastrozole) 3 days after their TC dosage. Anastrozole will prevent the conversion of TC to estradiol when taken between 48-72 hours post-TC injection. The half-life of anastrozole in humans ranges from 30-60 hours so timing the dosage of anastrozole is essential to maintaining consistent TT levels. Patients appeared to experience less side effects when they remained consistent with the timing and dosage of their TC and anastrozole. Patients frequently experienced acne, mood swings, and elevating hemoglobin when they did not stay consistent with both TC and anastrozole dosing. This strongly suggests that the fluctuation in either testosterone or estrogen levels can cause the unwanted side effects experienced with TRT.

Estrogen control was a vital part of the patients' stabilization of treatment. Patients' average estradiol level at stabilization for patients previously on treatment was 17.50 pg/ml and for patients never on treatment was 18.21 pg/ml. The average anastrozole dosage at stabilization for patients previously on treatment was 0.90 mg and for patients never on treatment was 0.75 mg. However, when patients were started on anastrozole at the beginning of treatment they frequently experienced hot flashes or joint pains as their estradiol would drop too low. Therefore, it is suggested that anastrozole be started once patients either begin experiencing bloating or their estradiol increases to >35 μg/ml. Stopping anastrozole completely was inappropriate because patients' TT levels would drop as a result of the conversion of TC to estradiol.

In this study, patients' TT levels stabilized between 605-1051 ng/dL, the point at which they no longer experienced the polycythemia. However, almost all patients were still experiencing symptoms of testicular hypofunction when their 7-day testosterone level was <600. Similarly, the side effects would start to outweigh the benefits of treatment when TT levels were >1050. Side effects seen at these higher levels included acne, aggression, erectile dysfunction, difficulty losing weight, extremely elevated estradiol levels, hair loss, and increased body hair. Hair loss with TRT did not occur at appropriate dosing and management unless patients had a strong family history of hair loss. In these cases, the hair loss tended to follow their family history as far as male pattern baldness. Similarly, patients were more likely to experience acne if they had a personal history of acne or naturally oily skin. Regardless, maintaining relatively consistent, appropriate TT and estradiol levels minimized these side effects and helped patients maintain consistency of treatment. Therefore, these data suggest the ideal target range for a patient's total testosterone level, drawn after 4 consecutive weeks of injections and drawn exactly 7 days after their previous injection, is 605-1051 ng/dL. Note that this is a much smaller range than the currently suggested range of 348-1197 ng/dL.

Additional benefits seen with TC and anastrozole dosing as described in this study include:
An average of 0.14% decrease in A1c for patients previously on TRT and 0.39% decrease in A1c for patients not previously on TRT.
Elimination of the common increase in cholesterol levels seen with excessive TC dosing.
An average weight loss of 1.03 lbs (0.44%) for patients never on TRT. However, those previously on TRT showed a slight increase (1.40 lbs or 0.64%) in weight on average.

These were secondary effects that were noticed when patients stabilized on treatment, but further studies will be needed to investigate the mechanism of action associated with the significant decrease in A1c.

Finally, PSA levels increased when starting TRT, as expected. No patients in this study developed prostate cancer, but there was a 14.36% increase in PSA on average from the start of treatment to stabilization in those who had previously been on treatment and a 26.89% increase in PSA for patients that had not previously been on treatment This was likely attributable to the androgen receptors present on the prostate, which will then slightly enlarge from the exposure to testosterone cypionate acting on these receptors.

Study Conclusion: Based on the data collected from 60 total patients that were able to reach stabilization on TRT (30 that had been on treatment previously and 30 that had never been on TRT), the average TC weekly dosage at which patients stabilized for those previously on TRT was 147 mg, and average TC weekly dosage at which patients stabilized for those who had never been on treatment was 149.3 mg. Therefore, it is recommended to start patients at 140 mg TC injections weekly (ideally every 7 days) for the first 4 weeks of treatment (accounting for the 30 day elimination time). The testosterone levels should then be re-measured at the half-life (7 days post-injection) for accuracy. We recommend dosing adjustments should be made in 20 mg increments and only after 4 consecutive injections of the same TC dose. TT levels stabilized at 780.1 ng/dL on average (range: 605-1020 ng/dL) for those previously on TRT and 794.53 ng/dL on average (range: 641-1051 ng/dL) for those never on TRT. Based on these results, the recommended target range for testosterone levels is 605-1051 ng/dL.

The only exceptions to these patterns were seen with patients in their low to mid-20's and patients over age 60. These 2 cohorts usually stabilized at a slightly lower dosage, suggesting that dosing for these patients should be started at 120 mg TC weekly injections. It is likely that patients in these 2 cohorts are experiencing less stress and more regular daily schedules than those in the middle of their careers, which may lead to a better response to the testosterone cypionate injections.

Estradiol levels at stabilization averaged 17.50 pg/ml (range: 5-37 pg/ml) for patients previously on TRT and 18.21 pg/ml (range: 5-39 pg/ml) for patients never on TRT. Patients previously on TRT were on an average anastrozole dosage of 0.90 mg at the time of stabilization and those never on TRT were on an average anastrozole dose of 0.75 mg at time of stabilization. Therefore, it is recommended to start dosing at 1 mg of anastrozole weekly, 3 days after TC injections. Patients in the study who experienced symptoms of low estrogen (hot flashes/joint pains/decreased libido) were then decreased to 0.5 mg of anastrozole weekly. Only 6 patients (10%) in the study were able to reach stabilization of treatment without taking any anastrozole.

A significant find of this study was that hemoglobin stabilization was vital to stabilization of treatment. Based on the results, it is recommended for patients to start 81 mg aspirin daily if their hemoglobin level approaches the upper portion of the normal range (17.1 g/dl) and maintain adequate hydration to help avoid the polycythemia. If their hemoglobin still elevates to >17 despite preventative measures, it is suggested to have the patient donate whole blood (or have 1 pint of blood taken as a therapeutic treatment in-office) to avoid risk of a blood clot. Patients with familial erythrocytosis whose hemoglobin starts at or above 17 g/dl are encouraged to donate blood and start 81 mg aspirin daily at the beginning of treatment. This will help to avoid the need to donate frequently, which can lead to iron deficiency and a return in their fatigue. The balance between donating blood appropriately and avoiding excessive donating can be difficult, but it is very possible when starting at the recommended TC and anastrozole doses with appropriate time intervals between doses.

In conclusion, it was noted that all patients who, when drawn seven days after an injection, after four consecutive injections, and when able to get to the TT and E2 ranges listed below, were able to reach stability. They were then able to prevent the need to donate blood by staying within those ranges. This was accomplished by being consistent with both their TC and anastrozole dosing.
TT: 605-1051 ng/dL
E2: 7-35 pg/mL

I claim:

1. A method of treating hypogonadism in a human male patient, the method comprising the steps of:
administering to a patient determined to be in need of such treatment a therapeutically effective amount of a testosterone androstane steroid derivative (TASD) sufficient to maintain a total serum testosterone level within about a therapeutic/stabilized range.

2. The method of claim 1, wherein the TASD is testosterone cypionate.

3. The method of claim 1, further comprising the step of:
if the patient is determined to have an elevated estrogen level, administering a therapeutically effective amount of an aromatase inhibitor sufficient to maintain an estradiol level at or below 35 μg/mL.

4. The method of claim 3, further comprising the step of:
if after administering the therapeutically effective amount of the aromatase inhibitor sufficient to maintain an estradiol level at or below 35 μg/mL, the patient is determined to have an estrogen level below a predetermined level, administering a revised therapeutically effective amount of the aromatase inhibitor sufficient to maintain the estradiol level above the predetermined level and the estradiol level at or below 35 μg/mL.

5. The method of claim 3, wherein the aromatase inhibitor is anastrozole.

6. The method of claim 1, further comprising the step of:
if the patient is determined to have an elevated hemoglobin level, administering a therapeutically effective amount of aspirin.

7. The method of claim 6, wherein the hemoglobin level is determined to be elevated if it is greater than about 16 g/dL

8. The method of claim 1, wherein the serum total testosterone level therapeutic/stabilized range is between about 605-1051 ng/dL.

9. A method of treating hypogonadism in a human male patient, the method comprising the steps of:
determining a first total serum testosterone level of the patient;
if the patient's first total serum testosterone level is below about 500 ng/dL, starting the patient on an initial dosage amount of about 140 mg of a testosterone androstane steroid derivative (TASD), administered about every 7 days;
after four consecutive TASD administrations, determining a second total serum testosterone level, an estrogen level, and a hemoglobin level of the patient;
if after four consecutive TASD administrations, the estrogen level is elevated, starting the patient on about 1 mg anastrozole, administered about once weekly:
if after taking the 1 mg anastrozole administered about once weekly the patient has a low estrogen level, decreasing the anastrozole dosage to about 0.5 mg, administered about once weekly;
if after the four consecutive TASD administrations the patient has an elevated hemoglobin level, starting the patient on aspirin, administered daily;
if after the four consecutive TASD administrations, the second total serum testosterone level is within a therapeutic/stabilized range and the four consecutive TASD administrations are therapeutically effective, continuing the patient on the initial dosage amount of the TASD;
if after the four consecutive TASD administrations, the second total serum testosterone level is above the therapeutic/stabilized range, decreasing the initial dosage amount of TASD by about 20 mg;
if after the four consecutive TASD administrations, the second total serum testosterone level is below the therapeutic/stabilized range, increasing the initial dosage amount of TASD by about 20 mg.

10. The method of claim 9, wherein the testosterone androstane steroid derivative is testosterone cypionate.

11. The method of claim 10, wherein the therapeutic/stabilized range is about 605-1051 ng/dL.

12. The method of claim 11, wherein the estrogen level is elevated if an estradiol level is greater than about 35 μg/mL.

13. The method of claim 11, wherein a determination is made that the estrogen level is elevated based on clinical indications.

14. The method of claim 11, wherein the hemoglobin level is elevated if the hemoglobin level is greater than about 16 g/dL.

15. The method of claim 11, wherein the blood is removed from the patient in therapeutic amounts if the hemoglobin level is greater than 17 g/dl.

16. The method of claim 9, further comprising the step of:
after the last step, determining subsequent total serum testosterone levels, and, using such subsequent total serum testosterone levels, administering the TASD in revised dosage amounts sufficient to maintain a steady state total serum testosterone level within about the therapeutic/stabilized range.

17. The method of claim 16, wherein the therapeutic/stabilized range is about 605-1051 ng/dL.

18. The method of claim 17, wherein the testosterone androstane steroid derivative is testosterone cypionate.

19. The method of claim 18, further comprising the step of:
after the last step, determining whether the patient has reached Stability.

20. The method of claim 19, wherein Stability is a point in time at which the presenting symptoms of the patient have resolved, polycythemia, if any, has stopped, and the testosterone cypionate dosage amounts and intervals remain consistent for at least about three months.
